(19) United States
(12) Patent Application Publication
(10) Pub. No.: US 2024/0175168 A1
(43) Pub. Date: May 30, 2024

(54) WICKING MATERIALS

(71) Applicant: Ohio State Innovation Foundation, Columbus, OH (US)

(72) Inventors: Ethan Hessick, Columbus, OH (US); Pelagia-Iren Gouma, Columbus, OH (US); Tessa Gilmore, Columbus, OH (US)

(21) Appl. No.: 18/524,669

(22) Filed: Nov. 30, 2023

Related U.S. Application Data
(60) Provisional application No. 63/428,856, filed on Nov. 30, 2022.

(51) Int. Cl. D01D 5/00; G01N 33/543

(52) U.S. Cl. CPC D01D 5/0038; G01N 33/54388; D10B 2201/28; D10B 2321/12

(57) ABSTRACT

Wicking materials are described comprising: a first layer comprising a plurality of cellulose acetate (CA) fibers; and a second layer comprising a plurality of polyvinylpyrrolidone (PVP) fibers. Methods of manufacturing the wicking materials and biosensors comprising the same are also described.

### CROSS-REFERENCE TO RELATED APPLICATIONS

This application claims the benefit of priority to U.S. Provisional Application No. 63/428,856 filed Nov. 30, 2022, the disclosure of which is incorporated herein by reference in its entirety.

### TECHNICAL FIELD

This disclosure relates to wicking materials, and more particularly to wicking materials comprising layers of cellulose acetate and polyvinylpyrrolidone fibers.

### BACKGROUND

Wicking materials are used in many biosensors, such as in lateral flow assays. The gold standard for wicking materials is those composed of nitrocellulose. However, nitrocellulose poses safety concerns due to its flammability. Thus, there is a clear need for different materials that are nonflammable that may be used in biosensors while still maintaining biocompatibility.

### SUMMARY

The present disclosure provides wicking materials that are nonflammable, as compared to nitrocellulose materials presently used, while still maintaining biocompatibility. The disclosed wicking materials may find use in such applications as biosensors, for example, in lateral flow assays.

Thus, in one aspect, a wicking material is provided comprising:
a first layer comprising a plurality of cellulose acetate (CA) fibers; and
a second layer comprising a plurality of polyvinylpyrrolidone (PVP) fibers.

In some aspects, the first layer is electrospun. In some aspects, the second layer is electrospun. In some aspects, the ratio of CA fibers to PVP fibers is from about 10:1 to about 1:10. In some aspects, the plurality of CA fibers has an average diameter from about 1 nm to about 10,000 nm. In some aspects, the plurality of PVP fibers has an average diameter from about 1 nm to about 10,000 nm.

In some aspects, the first layer exhibits porosity. In some aspects, the first layer has an average pore size from about 10 nm to about 100 μm. In some aspects, the second layer exhibits porosity. In some aspects, the second layer has an average pore size from about 10 nm to about 100 μm.

In some aspects, the wicking material described herein further comprises one or more detection agents. In some aspects, the one or more detection agents comprise an antibody. In some aspects, the one or more detection agents comprise a nanomaterial.

Also provided is a biosensor comprising a wicking material described herein. In some aspects, the biosensor comprises a lateral flow assay.

Also provided is a method of manufacturing a wicking material described herein comprising:
electrospinning a first layer comprising a plurality of cellulose acetate (CA) fibers from a CA melt or solution; and
electrospinning a second layer comprising a plurality of polyvinylpyrrolidone (PVP) fibers from a PVP melt or solution, wherein the second layer is electrospun upon the first layer.

The details of one or more aspects of the disclosure are set forth in the accompanying drawings and the description below. Other features, objects, and advantages of the disclosure will be apparent from the description, drawings, and claims.

### DESCRIPTION OF DRAWINGS

FIG. 1 is an overview of the development of the lateral flow immunoassay as described in the examples.

FIG. 2 describes sandwich immunoassay techniques for the detection of biomolecules via labeled antibodies using a flow assay using the wicking materials described herein.

FIG. 3 shows the electrospinning setup used in the preparation of the wicking materials described in the examples.

FIG. 4 is an SEM image of the wicking materials prepared in the examples.

FIG. 5 provides a representative sensor design using the wicking materials described herein.

FIG. 6 shows the confirmation of the color change mechanism for the control zone in the wicking material described in the examples.

FIG. 7 shows the release of antibodies when wicking occurs at the conjugate pad, as described in the examples.

Like reference symbols in the various drawings indicate like elements.

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain, benefiting from the teachings presented in the descriptions herein and the associated drawings. Therefore, it is understood that the disclosures are not limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As apparent to those of skill in the art upon reading this disclosure, each of the individual aspects described and illustrated herein has discrete components and features that may be readily separated from or combined with the features of any of the other several aspects without departing from the scope or spirit of the present disclosure.

Any recited method can be carried out in the order of events recited or any other order that is logically possible. Unless otherwise expressly stated, it is in no way intended that any method or aspect set forth herein be construed as requiring that its steps be performed in a specific order. Accordingly, where a method claim does not explicitly state in the claims or descriptions that the steps are to be limited to a particular order, it is in no way intended that an order be inferred in any respect. This holds for any possible non-express basis for interpretation, including logic concerning arrangement of steps or operational flow, meaning derived from grammatical organization or punctuation, or the number or type of aspects described in the specification.

All publications mentioned herein are incorporated by reference to disclose and describe the methods or materials in connection with which the publications are cited. The publications discussed herein are provided solely for their disclosure before the filing date of the present application. Nothing herein is to be construed as an admission that the present invention is not entitled to antedate such publication by prior invention. Further, the dates of publication provided herein can be different from the actual publication dates, which can require independent confirmation.

It is also to be understood that the terminology herein describes particular aspects only and is not intended to be limiting. Unless defined otherwise, all technical and scientific terms herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Before describing the various aspects of the present disclosure, the following definitions are provided and should be used unless otherwise indicated. Additional terms may be defined elsewhere in the present disclosure.

As used herein, “comprising” is interpreted as specifying the presence of the stated features, integers, steps, or components but does not preclude the presence or addition of one or more features, integers, steps, components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, “consisting essentially of” is intended to include examples encompassed by the term “consisting of.”

As used in the specification and the appended claims, the singular forms “a,” “an,” and “the” include plural referents unless the context dictates otherwise. Thus, for example, reference to “a layer,” “a material,” or “a biosensor” includes, but is not limited to, two or more such layers, materials, biosensors, and the like.

Ratios, concentrations, amounts, and other numerical data can be expressed herein in a range format. Further, the endpoints of each of the ranges are significant both in relation to the other endpoint and independently of the other endpoint. There are many values disclosed herein, and each value is also disclosed as “about” that particular value in addition to the value itself. For example, if the value “10” is disclosed, then “about 10” is also disclosed. Ranges can be expressed herein as from “about” one particular value and to “about” another particular value. Similarly, when values are expressed as approximations, using the antecedent “about,” the particular value forms a further aspect. For example, if the value “about 10” is disclosed, then “10” is also disclosed.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x,’ ‘about y,’ and ‘about z’ as well as the ranges of ‘less than x,’ ‘less than y.’ and ‘less than z.’ Likewise, the phrase ‘about x, y, z, or greater’ should be interpreted to include the specific ranges of ‘about x,’ ‘about y,’ and ‘about z’ as well as the ranges of ‘greater than x,’ greater than y,′ and ‘greater than z.’ In addition, the phrase “about ‘x’ to ‘y’,” where ‘x’ and ‘y’ are numerical values, includes “about ‘x’ to about ‘y’.”

Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5% but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, amounts, sizes, formulations, parameters, and other quantities and characteristics are not and need not be exact but may be approximate, larger or smaller, as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In such cases, as used herein, “about” and “at or about” mean the nominal value indicated ±10% variation unless otherwise indicated or inferred. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

As used herein, “optional” or “optionally” means that the subsequently described event or circumstance can or cannot occur. The description includes instances where said event or circumstance occurs and those where it does not.

The present disclosure provides wicking materials that are nonflammable, as compared to nitrocellulose materials presently used, while still maintaining biocompatibility. The disclosed wicking materials may find use in such applications as biosensors, for example, in lateral flow assays. The presently disclosed wicking materials show clear advantages over nitrocellulose materials currently used in terms of reduced flammability while still retaining biocompatibility, which is desired in particular applications, such as biosensors described herein.

Thus, in one aspect, a wicking material is provided comprising:
a first layer comprising a plurality of cellulose acetate (CA) fibers; and
a second layer comprising a plurality of polyvinylpyrrolidone (PVP) fibers.

In some aspects, the wicking materials described herein are nonwoven, i.e., do not comprise a fabric in the sense of a material made by organizing the fibers into warp threads and weft threads.

“Cellulose acetate” refers to any esterified cellulosic polymer (which may also be referred to as a “polymer of glucose” or “polysaccharidic”), natural or synthetic, esterified preferably with acetic acid but without excluding other esterifying groups such as propionate or butyrate. In general, cellulose acetate, as used herein, is “untreated” in the sense that no provision is made to chemically alter the hydrophilicity or other properties of the cellulose acetate. The term “untreated” is not intended, however, to preclude the processing of the fibers therein for other purposes. Thus, the fibers and wicking materials from which they are made may contain (by way of example and not of limitation) metals, metal oxides, organic or inorganic dyestuff, etc.

The first layer and second layers of the wicking material may be prepared by any appropriate method known to those skilled in the art but are typically prepared by electrospinning. In electrospinning, a high voltage (e.g., about 3 to about 50 kV) is applied between a target (or collector) and a conducting capillary into which a polymer solution or melt is injected. The high voltage can also be applied to the solution or melt through a wire if the capillary is a nonconductor, such as a glass pipette. The collector may be a metal plate or screen, a rotating drum, or even a liquid bath if the capillary is vertical. Initially, the solution at the open tip of the capillary is pulled into a conical shape (the so-called “Taylor cone”) through the interplay of electrical force and surface tension. At a certain voltage range, a fine jet of polymer solution (or melt) forms at the tip of the Taylor cone and shoots toward the target. Forces from the electric field accelerate and stretch the jet. This stretching, together with the evaporation of solvent molecules, causes the jet diameter to become smaller. As the jet diameter decreases, the charge density increases until electrostatic forces within the polymer overcome the cohesive forces holding the jet together (e.g., surface tension), causing the jet to split or “splay” into a multifilament of polymer fibers. The fibers continue to splay until they reach the collector, where they are collected as nonwoven fibers and are optionally dried. In some aspects, the diameter of the electrospun nanofiber may typically be between about 50 nm and about 5 μm.

Electrospinning is a well-known method of fabricating thin threads or fibers from dissolved polymers. In one aspect, the polymer solution (the “precursor” of the nanofibers) is expressed from a syringe driven by a syringe pump. The solution is forced through a hollow needle and exits as tiny droplets. Each droplet immediately traverses a field of high voltage. The potential applied to the solution as it emerges from the needle tip induces an accumulation of charges on the surface of the droplet, which changes the surface tension of the droplet, causing the surface to “break” such that the droplet becomes a jet stream of charged fibers that can be collected as a charged active matrix, which can build up to form the desired layer. Any surface that is “at ground” relative to the potential on a droplet whose surface has just been charged in an electric field can serve as a “collector” for the spun fibers.

Adjustments to the properties of the electric field, the concentration of polymer in the precursor solution, the solvent and the polymer used, the pressure and flow rate of the precursor solution from the needle tip, the distance from the needle tip to the collection surface, and ambient conditions (temperature, pressure, ambient gases) allow persons of skill in the art to generate fibers of predetermined thickness at predetermined rates to build up mats of predetermined density, porosity, and thickness.

In some aspects, the first layer is electrospun. In some aspects, the second layer is electrospun. In some aspects, both the first layer and the second layer are electrospun.

In some aspects, the ratio of CA fibers to PVP fibers is from about 10:1 to about 1:10, for example, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, or about 1:10.

In some aspects, the plurality of CA fibers have an average diameter from about 1 nm to about 10,000 nm, for example, from about 1 nm to about 10 nm, from about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 1 nm to about 100 nm, from about 1 nm to about 200 nm, from about 1 nm to about 300 nm, from about 1 nm to about 400 nm, from about 1 nm to about 500 nm, from about 1 nm to about 1,000 nm, from about 1 nm to about 5,000 nm, from about 1 nm to about 10,000 nm, from about 10 nm to about 20 nm, from about 10 nm to about 30 nm, from about 10 nm to about 40 nm, from about 10 nm to about 50 nm, from about 10 nm to about 60 nm, from about 10 nm to about 70 nm, from about 10 nm to about 80 nm, from about 10 nm to about 90 nm, from about 10 nm to about 100 nm, from about 10 nm to about 200 nm, from about 10 nm to about 300 nm, from about 10 nm to about 400 nm, from about 10 nm to about 500 nm, from about 10 nm to about 1,000 nm, from about 10 nm to about 5,000 nm, from about 10 nm to about 10,000 nm, from about 20 nm to about 30 nm, from about 20 nm to about 40 nm, from about 20 nm to about 50 nm, from about 20 nm to about 60 nm, from about 20 nm to about 70 nm, from about 20 nm to about 80 nm, from about 20 nm to about 90 nm, from about 20 nm to about 100 nm, from about 20 nm to about 200 nm, from about 20 nm to about 300 nm, from about 20 nm to about 400 nm, from about 20 nm to about 500 nm, from about 20 nm to about 1,000 nm, from about 20 nm to about 5,000 nm, from about 20 nm to about 10,000 nm, from about 30 nm to about 40 nm, from about 30 nm to about 50 nm, from about 30 nm to about 60 nm, from about 30 nm to about 70 nm, from about 30 nm to about 80 nm, from about 30 nm to about 90 nm, from about 30 nm to about 100 nm, from about 30 nm to about 200 nm, from about 30 nm to about 300 nm, from about 30 nm to about 400 nm, from about 30 nm to about 500 nm, from about 30 nm to about 1,000 nm, from about 30 nm to about 5,000 nm, from about 30 nm to about 10,000 nm, from about 40 nm to about 50 nm, from about 40 nm to about 60 nm, from about 40 nm to about 70 nm, from about 40 nm to about 80 nm, from about 40 nm to about 90 nm, from about 40 nm to about 100 nm, from about 40 nm to about 200 nm, from about 40 nm to about 300 nm, from about 40 nm to about 400 nm, from about 40 nm to about 500 nm, from about 40 nm to about 1,000 nm, from about 40 nm to about 5,000 nm, from about 40 nm to about 10,000 nm, from about 50 nm to about 60 nm, from about 50 nm to about 70 nm, from about 50 nm to about 80 nm, from about 50 nm to about 90 nm, from about 50 nm to about 100 nm, from about 50 nm to about 200 nm, from about 50 nm to about 300 nm, from about 50 nm to about 400 nm, from about 50 nm to about 500 nm, from about 50 nm to about 1,000 nm, from about 50 nm to about 5,000 nm, from about 50 nm to about 10,000 nm, from about 60 nm to about 70 nm, from about 60 nm to about 80 nm, from about 60 nm to about 90 nm, from about 60 nm to about 100 nm, from about 60 nm to about 200 nm, from about 60 nm to about 300 nm, from about 60 nm to about 400 nm, from about 60 nm to about 500 nm, from about 60 nm to about 1,000 nm, from about 60 nm to about 5,000 nm, from about 60 nm to about 10,000 nm, from about 70 nm to about 80 nm, from about 70 nm to about 90 nm, from about 70 nm to about 100 nm, from about 70 nm to about 200 nm, from about 70 nm to about 300 nm, from about 70 nm to about 400 nm, from about 70 nm to about 500 nm, from about 70 nm to about 1,000 nm, from about 70 nm to about 5,000 nm, from about 70 nm to about 10,000 nm, from about 80 nm to about 90 nm, from about 80 nm to about 100 nm, from about 80 nm to about 200 nm, from about 80 nm to about 300 nm, from about 80 nm to about 400 nm, from about 80 nm to about 500 nm, from about 80 nm to about 1,000 nm, from about 80 nm to about 5,000 nm, from about 80 nm to about 10,000 nm, from about 90 nm to about 100 nm, from about 90 nm to about 200 nm, from about 90 nm to about 300 nm, from about 90 nm to about 400 nm, from about 90 nm to about 500 nm, from about 90 nm to about 1,000 nm, from about 90 nm to about 5,000 nm, from about 90 nm to about 10,000 nm, from about 100 nm to about 200 nm, from about 100 nm to about 300 nm, from about 100 nm to about 400 nm, from about 100 nm to about 500 nm, from about 100 nm to about 1,000 nm, from about 100 nm to about 5,000 nm, from about 100 nm to about 10,000 nm, from about 200 nm to about 300 nm, from about 200 nm to about 400 nm, from about 200 nm to about 500 nm, from about 200 nm to about 1,000 nm, from about 200 nm to about 5,000 nm, from about 200 nm to about 10,000 nm, from about 300 nm to about 400 nm, from about 300 nm to about 500 nm, from about 300 nm to about 1,000 nm, from about 300 nm to about 5,000 nm, from about 300 nm to about 10,000 nm, from about 400 nm to about 500 nm, from about 400 nm to about 1,000 nm, from about 400 nm to about 5,000 nm, from about 400 nm to about 10,000 nm, from about 500 nm to about 1,000 nm, from about 500 nm to about 5,000 nm, from about 500 nm to about 10,000 nm, from about 1,000 nm to about 5,000 nm, from about 1,000 nm to about 10,000 nm, or from about 5,000 nm to about 10,000 nm.

In some aspects, the plurality of PVP fibers have an average diameter from about 1 nm to about 10,000 nm, for example, from about 1 nm to about 10 nm, from about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 1 nm to about 100 nm, from about 1 nm to about 200 nm, from about 1 nm to about 300 nm, from about 1 nm to about 400 nm, from about 1 nm to about 500 nm, from about 1 nm to about 1,000 nm, from about 1 nm to about 5,000 nm, from about 1 nm to about 10,000 nm, from about 10 nm to about 20 nm, from about 10 nm to about 30 nm, from about 10 nm to about 40 nm, from about 10 nm to about 50 nm, from about 10 nm to about 60 nm, from about 10 nm to about 70 nm, from about 10 nm to about 80 nm, from about 10 nm to about 90 nm, from about 10 nm to about 100 nm, from about 10 nm to about 200 nm, from about 10 nm to about 300 nm, from about 10 nm to about 400 nm, from about 10 nm to about 500 nm, from about 10 nm to about 1,000 nm, from about 10 nm to about 5,000 nm, from about 10 nm to about 10,000 nm, from about 20 nm to about 30 nm, from about 20 nm to about 40 nm, from about 20 nm to about 50 nm, from about 20 nm to about 60 nm, from about 20 nm to about 70 nm, from about 20 nm to about 80 nm, from about 20 nm to about 90 nm, from about 20 nm to about 100 nm, from about 20 nm to about 200 nm, from about 20 nm to about 300 nm, from about 20 nm to about 400 nm, from about 20 nm to about 500 nm, from about 20 nm to about 1,000 nm, from about 20 nm to about 5,000 nm, from about 20 nm to about 10,000 nm, from about 30 nm to about 40 nm, from about 30 nm to about 50 nm, from about 30 nm to about 60 nm, from about 30 nm to about 70 nm, from about 30 nm to about 80 nm, from about 30 nm to about 90 nm, from about 30 nm to about 100 nm, from about 30 nm to about 200 nm, from about 30 nm to about 300 nm, from about 30 nm to about 400 nm, from about 30 nm to about 500 nm, from about 30 nm to about 1,000 nm, from about 30 nm to about 5,000 nm, from about 30 nm to about 10,000 nm, from about 40 nm to about 50 nm, from about 40 nm to about 60 nm, from about 40 nm to about 70 nm, from about 40 nm to about 80 nm, from about 40 nm to about 90 nm, from about 40 nm to about 100 nm, from about 40 nm to about 200 nm, from about 40 nm to about 300 nm, from about 40 nm to about 400 nm, from about 40 nm to about 500 nm, from about 40 nm to about 1,000 nm, from about 40 nm to about 5,000 nm, from about 40 nm to about 10,000 nm, from about 50 nm to about 60 nm, from about 50 nm to about 70 nm, from about 50 nm to about 80 nm, from about 50 nm to about 90 nm, from about 50 nm to about 100 nm, from about 50 nm to about 200 nm, from about 50 nm to about 300 nm, from about 50 nm to about 400 nm, from about 50 nm to about 500 nm, from about 50 nm to about 1,000 nm, from about 50 nm to about 5,000 nm, from about 50 nm to about 10,000 nm, from about 60 nm to about 70 nm, from about 60 nm to about 80 nm, from about 60 nm to about 90 nm, from about 60 nm to about 100 nm, from about 60 nm to about 200 nm, from about 60 nm to about 300 nm, from about 60 nm to about 400 nm, from about 60 nm to about 500 nm, from about 60 nm to about 1,000 nm, from about 60 nm to about 5,000 nm, from about 60 nm to about 10,000 nm, from about 70 nm to about 80 nm, from about 70 nm to about 90 nm, from about 70 nm to about 100 nm, from about 70 nm to about 200 nm, from about 70 nm to about 300 nm, from about 70 nm to about 400 nm, from about 70 nm to about 500 nm, from about 70 nm to about 1,000 nm, from about 70 nm to about 5,000 nm, from about 70 nm to about 10,000 nm, from about 80 nm to about 90 nm, from about 80 nm to about 100 nm, from about 80 nm to about 200 nm, from about 80 nm to about 300 nm, from about 80 nm to about 400 nm, from about 80 nm to about 500 nm, from about 80 nm to about 1,000 nm, from about 80 nm to about 5,000 nm, from about 80 nm to about 10,000 nm, from about 90 nm to about 100 nm, from about 90 nm to about 200 nm, from about 90 nm to about 300 nm, from about 90 nm to about 400 nm, from about 90 nm to about 500 nm, from about 90 nm to about 1,000 nm, from about 90 nm to about 5,000 nm, from about 90 nm to about 10,000 nm, from about 100 nm to about 200 nm, from about 100 nm to about 300 nm, from about 100 nm to about 400 nm, from about 100 nm to about 500 nm, from about 100 nm to about 1,000 nm, from about 100 nm to about 5,000 nm, from about 100 nm to about 10,000 nm, from about 200 nm to about 300 nm, from about 200 nm to about 400 nm, from about 200 nm to about 500 nm, from about 200 nm to about 1,000 nm, from about 200 nm to about 5,000 nm, from about 200 nm to about 10,000 nm, from about 300 nm to about 400 nm, from about 300 nm to about 500 nm, from about 300 nm to about 1,000 nm, from about 300 nm to about 5,000 nm, from about 300 nm to about 10,000 nm, from about 400 nm to about 500 nm, from about 400 nm to about 1,000 nm, from about 400 nm to about 5,000 nm, from about 400 nm to about 10,000 nm, from about 500 nm to about 1,000 nm, from about 500 nm to about 5,000 nm, from about 500 nm to about 10,000 nm, from about 1,000 nm to about 5,000 nm, from about 1,000 nm to about 10,000 nm, or from about 5,000 nm to about 10,000 nm.

Nanofiber diameters ranging from about 1 micrometer to about 1 nanometer may be useful in certain aspects of the disclosure. Generally, a range from about 1 micrometer to about 10 nanometers is preferred. A range from about 50 to 500 nanometers is more preferred, and a range from about 100 to 300 nanometers is most preferred. An environment of air comprising gases at about standard partial pressures and temperatures (0-30° C.) is suitable for generating the nanofibers used in aspects of the invention, but higher temperatures, such as those used for thermoset processes, are not to be excluded. Neither are non-standard mixtures of air gases, gases not normally present in the air, or non-standard pressures.

In some aspects, the first layer exhibits porosity. In some aspects, the first layer has an average pore size from about 10 nm to about 100 μm, for example, from about 10 nm to about 100 nm, from about 10 nm to about 500 nm, from about 10 nm to about 1 μm, from about 10 nm to about 5 μm, from about 10 nm to about 10 μm, from about 10 nm to about 50 μm, from about 10 nm to about 100 μm, from about 100 nm to about 500 nm, from about 100 nm to about 1 μm, from about 100 nm to about 5 μm, from about 100 nm to about 10 μm, from about 100 nm to about 50 μm, from about 100 nm to about 100 μm, from about 500 nm to about 1 μm, from about 500 nm to about 5 μm, from about 500 nm to about 10 μm, from about 500 nm to about 50 μm, from about 500 nm to about 100 μm, from about 1 μm to about 5 μm, from about 1 μm to about 10 μm, from about 1 μm to about 50 μm, from about 1 μm to about 100 μm, from about 5 μm to about 10 μm, from about 5 μm to about 50 μm, from about 5 μm to about 100 μm, from about 10 μm to about 50 μm, from about 10 μm to about 100 μm, or from about 50 μm to about 100 μm.

In some aspects, the second layer exhibits porosity. In some aspects, the second layer has an average pore size from about 10 nm to about 100 μm, for example, from about 10 nm to about 100 nm, from about 10 nm to about 500 nm, from about 10 nm to about 1 μm, from about 10 nm to about 5 μm, from about 10 nm to about 10 μm, from about 10 nm to about 50 μm, from about 10 nm to about 100 μm, from about 100 nm to about 500 nm, from about 100 nm to about 1 μm, from about 100 nm to about 5 μm, from about 100 nm to about 10 μm, from about 100 nm to about 50 μm, from about 100 nm to about 100 μm, from about 500 nm to about 1 μm, from about 500 nm to about 5 μm, from about 500 nm to about 10 μm, from about 500 nm to about 50 μm, from about 500 nm to about 100 μm, from about 1 μm to about 5 μm, from about 1 μm to about 10 μm, from about 1 μm to about 50 μm, from about 1 μm to about 100 μm, from about 5 μm to about 10 μm, from about 5 μm to about 50 μm, from about 5 μm to about 100 μm, from about 10 μm to about 50 μm, from about 10 μm to about 100 μm, or from about 50 μm to about 100 μm.

In some aspects, the wicking material further comprises one or more detection agents. In some aspects, the one or more detection agents comprise an antibody. Suitable antibodies that may be used as detection agents include, but are not limited to, those described in US 2021/0145736, incorporated herein by reference in its entirety. The terms “antibody” and “immunoglobulin” include antibodies and immunoglobulins of any isotope (e.g., IgG (e.g., IgG1, IgG2, IgG3, or IgG4), IgE, IgD, IgA, IgM, etc.), whole antibodies (e.g., antibodies composed of a tetramer which in term is composed of two dimers of a heavy and light chain polypeptide); single chain antibodies; fragments of antibodies (e.g., fragments of whole or single chain antibodies) which retain specific binding to the cell surface molecule or extracellular molecule, including, but not limited to, Fv, single chain Fv (scFv), Fab, F(ab′)₂, Fab′, (scFv′)₂, diabodies, and nanobodies; chimeric antibodies; monoclonal antibodies; fully human antibodies; humanized antibodies (e.g., humanized whole antibodies, humanized antibody fragments, etc.); and fusion proteins including an antigen-binding portion of an antibody and a non-antibody protein or fragment thereof. The antibody may be detectably labeled, e.g., with an in vivo imaging agent or the like. The antibody may be further conjugated to other moieties, such as polyethylene glycol (PEG) etc.

In some aspects, the one or more detection agents comprise a nanomaterial. Representative examples of such materials include but are not limited to, those described in US 2009/0110926, US 2011/0061446, and US 2013/0115706, each of which is incorporated herein by reference in their entirety.

In another aspect, a biosensor is provided comprising a wicking material as described herein. In some aspects, the biomaterial comprises a later flow assay.

In another aspect, a method of manufacturing a wicking material described herein, the method comprising:
electrospinning a first layer comprising a plurality of cellulose acetate (CA) fibers from a CA melt or solution; and
electrospinning a second layer comprising a plurality of polyvinylpyrrolidone (PVP) fibers from a PVP melt or solution, wherein the second layer is electrospun upon the first layer.

In view of the described materials and processes, hereinbelow are described certain more particularly described aspects of the disclosures. These particularly recited aspects should not, however, be interpreted to have any limiting effect on any different claims containing different or more general teachings described herein or that the “particular” aspects are somehow limited in some way other than the inherent meanings of the language and formulas literally used therein.
Aspect 1. A wicking material comprising:
   a first layer comprising a plurality of cellulose acetate (CA) fibers; and
   a second layer comprising a plurality of polyvinylpyrrolidone (PVP) fibers.
Aspect 2. The wicking material of aspect 1, wherein the first layer is electrospun.
Aspect 3. The wicking material of aspect 1 or aspect 2, wherein the second layer is electrospun.
Aspect 4. The wicking material of any one of aspects 1-3, wherein the ratio of CA fibers to PVP fibers is from about 10:1 to about 1:10.
Aspect 5. The wicking material of any one of aspects 1-4, wherein the plurality of CA fibers have an average diameter from about 1 nm to about 10,000 nm.
Aspect 6. The wicking material of any one of aspects 1-5, wherein the plurality of PVP fibers have an average diameter from about 1 nm to about 10,000 nm.
Aspect 7. The wicking material of any one of aspects 1-6, wherein the first layer exhibits porosity.
Aspect 8. The wicking material of aspect 7, wherein the first layer has an average pore size from about 10 nm to about 100 μm.
Aspect 9. The wicking material of any one of aspects 1-8, wherein the second layer exhibits porosity.
Aspect 10. The wicking material of aspect 9, wherein the second layer has an average pore size from about 10 nm to about 100 μm.
Aspect 11. The wicking material of any one of aspects 1-10, further comprising one or more detection agents.
Aspect 12. The wicking material of aspect 11, wherein the one or more detection agents comprise an antibody.
Aspect 13. The wicking material of aspect 11, wherein the one or more detection agents comprise a nanomaterial.
Aspect 14. A biosensor comprising a wicking material of any one of aspects 1-13.
Aspect 15. The biosensor of claim 14, wherein the biosensor comprises a lateral flow assay.
Aspect 16. A method of manufacturing a wicking material of any one of aspects 1-13, the method comprising:
   electrospinning a first layer comprising a plurality of cellulose acetate (CA) fibers from a CA melt or solution; and
   electrospinning a second layer comprising a plurality of polyvinylpyrrolidone (PVP) fibers from a PVP melt or solution, wherein the second layer is electrospun upon the first layer.

A number of aspects of the disclosure have been described. Nevertheless, it will be understood that various modifications may be made without departing from the spirit and scope of the invention. Accordingly, other aspects are within the scope of the following claims.

### Examples

The following examples are put forth to provide those of ordinary skill in the art with a complete disclosure and description of how the compositions, articles, devices, and methods claimed herein are made and evaluated and are intended to be purely exemplary of the invention and are not intended to limit the scope of what the inventors regard as their invention. Efforts have been made to ensure accuracy concerning numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

### Development of a Lateral Flow Immunoassay for Detection of Harmful Allergens Found in Dust

A total of 8% of the US population suffers from asthma (see L. J. Akinbami, J. E. Moorman, P. L. Garbe, and E. J. Sondik, “Status of childhood asthma in the United States, 1980-2007,” Pediatrics, vol. 123 Suppl 3, pp. S131-145, March 2009). Prior research has shown that specific allergens from house pests, including mice (see E. N. Torjusen, G. B. Diette, P. N. Breysse, J. Curtin-Brosnan, C. Aloe, and E. C. Matsui, “Dose-response relationships between mouse allergen exposure and asthma morbidity among urban children and adolescents,” Indoor Air, vol. 23, no. 4, pp. 268-274, August 2013), dust mites (see R. Sporik, S. T. Holgate, T. A. Platts-Mills, and J. J. Cogswell, “Exposure to house-dust mite allergen (Der p I) and the development of asthma in childhood. A prospective study,” N. Engl. J. Med., vol. 323, no. 8, pp. 502-507, August 1990), and cockroaches (see B. P. Leaderer et al., “Dust mite, cockroach, cat, and dog allergen concentrations in homes of asthmatic children in the northeastern United States: impact of socioeconomic factors and population density,” Environ. Health Perspect., vol. 110, no. 4, pp. 419-425, April 2002), can lead to worsened symptoms for asthmatic patients. The purpose of this project, shown in FIG. 1, is to make a point-of-care sensor for the detection of these allergens using a novel material system and phone app. If triggers can be identified, cost-effective solutions are available to the patient (see M. Kattan et al., “Cost-effectiveness of a home-based environmental intervention for inner-city children with asthma,” J. Allergy Clin. Immunol., vol. 116, no. 5, pp. 1058-1063, November 2005). These solutions have been shown to reduce asthma symptoms in patients without the use of medication or side effects (see D. D. Crocker et al., Effectiveness of home-based, multi-trigger, multicomponent interventions with an environmental focus for reducing asthma morbidity: a community guide systematic review. Centre for Reviews and Dissemination (UK), 2011. Accessed: Jan. 25, 2022. [Online]. Available: http://www.ncbi.nlm.nih.gov/books/NBK81131/; and W. J. Morgan et al., “Results of a home-based environmental intervention among urban children with asthma,” N. Engl. J. Med., vol. 351, no. 11, pp. 1068-1080, September 2004).

### Methods

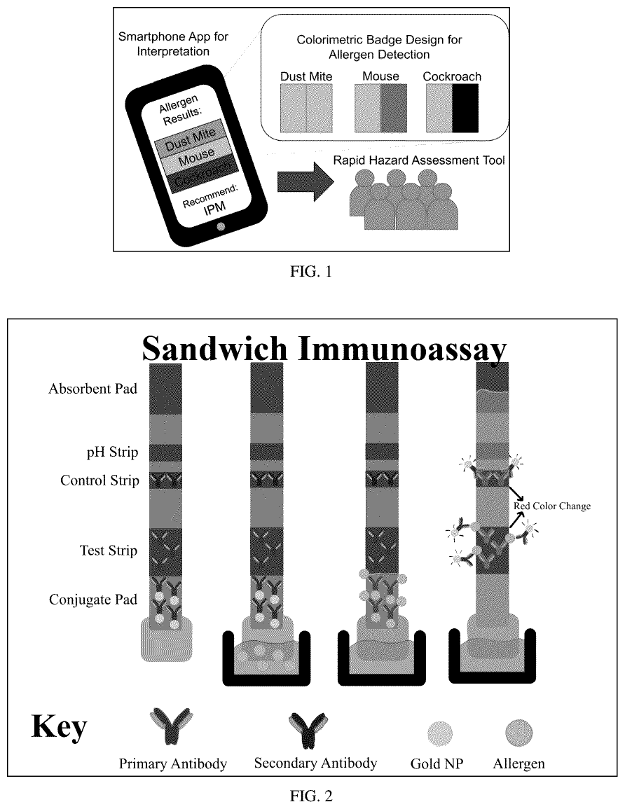

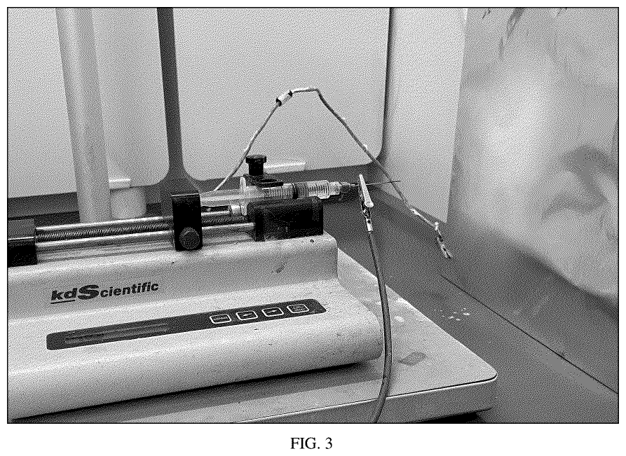

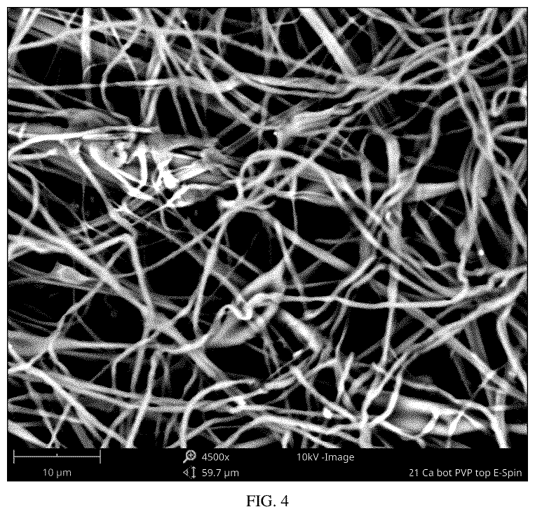

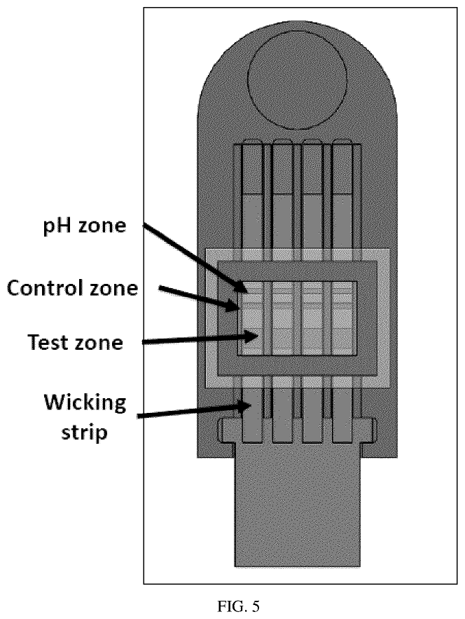

Electrospinning is used to create a composite layered polymer material for the different parts of the sensor strips, as described herein. FIGS. 3 and 4 show the electrospinning setup used and the resulting nanofibers. In-lab imaging is performed using a Phenom SEM. The sandwich detection technique shown in FIG. 2 is being tested for producing the color change (see E. B. Bahadιr and M. K. Sezgintürk, “Lateral flow assays: Principles, designs and labels,” TrAC Trends in Analytical Chemistry, vol. 82, pp. 286-306, September 2016). Each part of the strip is produced from polymer materials. Treatment steps, including heating, drying, and chemical modification via salts and/or blocking proteins, are used to modify the functionality of the polymer materials to suit each relevant strip region (see “Equipment-free, salt-mediated immobilization of nucleic acids for nucleic acid lateral flow assays,” Sensors and Actuators B: Chemical, vol. 351, p. 130975, January 2022; and L. Zhan et al., “Development and optimization of thermal contrast amplification lateral flow immunoassays for ultrasensitive HIV p24 protein detection,” Microsyst Nanoeng, vol. 6, no. 1, Art. no. 1, July 2020).

### Results

Wicking: The wicking performance of the electrospun composite polymer has been compared to the industry standard nitrocellulose. Our composite polymer can vertically wick within the time frame to conduct a POC test.

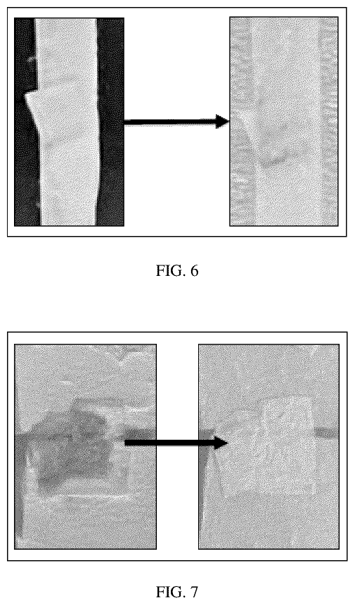

Proof of Concept: FIG. 6 shows the color change produced by an initial experiment to validate the material system and control strip detection method.

Test/Control Zone: The test and control zone materials must have immobilized antibodies. Through the addition of salt treatment and heating/drying steps, the electrospun polymer is more effective at immobilizing antibodies.

Conjugate Pad: The conjugate pad must release labeled antibodies when wicked. Pretreatment of electrospun polymer material with blocking proteins has produced effective conjugate pad materials that readily release antibodies when wicking occurs. FIG. 7 shows an exemplary conjugate pad.

### CONCLUSIONS

It is possible to produce wicking materials using electrospinning. These materials are not as hazardous as nitrocellulose. Sandwich immunoassay technique causes color change with our novel material system. Each part of a lateral flow assay can be produced via modification of initial wicking membrane materials. Once finalized, this technology will be adaptable to detect a wide variety of diseases and molecules. Different biomolecule labels can be tested to optimize the detection range and device sensitivity. The amount of antibody present in each strip can be optimized to produce a color change in the relevant allergen detection zone. The corresponding color change for given allergen concentrations can be recorded for calibration with the app. Strips can be mass-produced and tested in patients' homes (see A. K. Yetisen, M. S. Akram, and C. R. Lowe, “Paper-based microfluidic point-of-care diagnostic devices,” Lab Chip, vol. 13, no. 12, pp. 2210-2251).

The compositions and methods of the appended claims are not limited in scope by the specific compositions and methods described herein, which are intended as illustrations of a few aspects of the claims, and any compositions and methods that are functionally equivalent are intended to fall within the scope of the claims. Various modifications of the compositions and methods, in addition to those shown and described herein, are intended to fall within the scope of the appended claims. Further, while only certain representative compositions and method steps disclosed herein are specifically described, other combinations of the compositions and method steps are also intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

What is claimed is:

1. A wicking material comprising:
a first layer comprising a plurality of cellulose acetate (CA) fibers; and
a second layer comprising a plurality of polyvinylpyrrolidone (PVP) fibers.

2. The wicking material of claim 1, wherein the first layer is electrospun.

3. The wicking material of claim 1, wherein the second layer is electrospun.

4. The wicking material of claim 1, wherein the ratio of CA fibers to PVP fibers is from about 10:1 to about 1:10.

5. The wicking material of claim 1, wherein the plurality of CA fibers have an average diameter from about 1 nm to about 10,000 nm.

6. The wicking material of claim 1, wherein the plurality of PVP fibers have an average diameter from about 1 nm to about 10,000 nm.

7. The wicking material of claim 1, wherein the first layer exhibits porosity.

8. The wicking material of claim 7, wherein the first layer has an average pore size from about 10 nm to about 100 μm.

9. The wicking material of claim 1, wherein the second layer exhibits porosity.

10. The wicking material of claim 9, wherein the second layer has an average pore size from about 10 nm to about 100 μm.

11. The wicking material of claim 1, further comprising one or more detection agents.

12. The wicking material of claim 11, wherein the one or more detection agents comprise an antibody.

13. The wicking material of claim 11, wherein the one or more detection agents comprise a nanomaterial.

14. A biosensor comprising a wicking material of claim 1.

15. The biosensor of claim 14, wherein the biosensor comprises a lateral flow assay.

16. A method of manufacturing a wicking material of claim 1, the method comprising:
electrospinning a first layer comprising a plurality of cellulose acetate (CA) fibers from a CA melt or solution; and
electrospinning a second layer comprising a plurality of polyvinylpyrrolidone (PVP) fibers from a PVP melt or solution, wherein the second layer is electrospun upon the first layer.
